(19) United States
(12) Patent Application Publication
(10) Pub. No.: US 2015/0268237 A1
(43) Pub. Date: Sep. 24, 2015

(54) BIOASSAY SYSTEM AND METHOD FOR DETECTING ANALYTES IN BODY FLUIDS

(71) Applicants: Josef Kerimo, Concord, MA (US); Hansong Zeng, Acton, MA (US); Ron Scharlack, Brookline, MA (US); Gert Blankenstein, Cambridge, MA (US)

(72) Inventors: Josef Kerimo, Concord, MA (US); Hansong Zeng, Acton, MA (US); Ron Scharlack, Brookline, MA (US); Gert Blankenstein, Cambridge, MA (US)

(21) Appl. No.: 14/663,560

(22) Filed: Mar. 20, 2015

Related U.S. Application Data
(60) Provisional application No. 61/969,371, filed on Mar. 24, 2014.

(51) Int. Cl. G01N 33/543; G01N 21/41

(52) U.S. Cl. CPC G01N 33/54373; G01N 33/54346; G01N 21/41

(57) ABSTRACT

A bioserisor platform is configured with upconverting nanoparticles on the surface of a resonant grating structure with enhanced sensitivity. The grating structure is illuminated with a light beam at one of its resonance modes to form a strong evanescent field at the surface and used for high sensitivity assays. The strong evanescent field triggers the upconverting nanoparticles to generate enhanced and localized emission on the grating surface, with lower background and lower auto-fluorescence from the grating substrate. This leads to improved performance in detecting analytes in bioassays.

### CROSS-REFERENCES TO RELATED APPLICATION

This application claims priority to and benefit of U.S. Provisional Application No. 61/969,371, filed Mar. 24, 2014, the entire contents of which are incorporated by reference herein for all purposes.

### TECHNICAL FIELD

The present invention relates to a bioassay system and a method for sensing analytes in a body fluid. More particularly, the present invention relates to a bioassay apparatus, for example, an immunoassay apparatus, comprising a resonance grating structure, and a method for detecting analytes in a body fluid using upconverting nanoparticles and the resonance grating structure.

### BACKGROUND

Traditionally, fluorophores are used as labels in bioassay apparatuses. However, fluorophores suffer photobleaching as time elapses. Various attempts have been made to use upconverting nanoparticles (i.e., particles having a diameter of between 1 and 100 nanometers and emitting light at a wavelength shorter than that of the excitation) as labels in bioassay apparatuses, because upconversing nanoparticles do not photobleach. However, the use of upconverting nanoparticles triggers other problems. For example, the emission from upconverting nanoparticles is rather weak due to the low quantum efficiency of upconverting nanoparticles and relatively high light levels are required for their excitation (e.g., 100 mW of 980 nm focused light). The upconverted emission is weak because the quantum efficiency (QE) of upconversion is typically less than 0.3%. In comparison, conventional fluorescence labels are much brighter because their QE can be greater than 20%. One approach for improving the upconversion emission is to chemically modify the nanoparticle surface but the success has been limited.

### SUMMARY OF THE INVENTION

Upconverting nanoparticles are promising fluorophores and/or labels in bioassays, because they provide an almost background-free detection system with substantially no photobleaching effects. “Upconverting” as used herein means the emission of light at a wavelength shorter than that of the excitation light. For example, the nanoparticles may absorb multiple near-infrared (NIR) photons (e.g., two or three photons at about 980 nm) and then emit green light (about 510 nm) or red light (about 650 nm) in the visible region. The “upconversion” process (i.e., emission at a wavelength shorter than that of the excitation) rarely occurs in biological samples. It can be observed with conventional continuous wave (CW) light sources (e.g., femto second pulsed NIR lasers with high peak power, causing high autofluorescence, are not required). Accordingly, a probing light beam would only trigger emission from the upconverting nanoparticles, and would almost never trigger emission from the biological samples. Therefore, the use of upconverting nanoparticles can lead to almost background-free detection when NIR CW light is used for the excitation and the blue-shifted emission is detected.

In bioassay applications, such background-free detection can lead to improvements in sensing capabilities. Typically, the background level determines the lower limit of detection in the assay and when it is too high the measurement precision suffers. In particular, UV light sources are commonly used in bioassays, which lead to high background from autofluorescence and scattering from the sample, and in the analysis of body fluid samples such as whole blood cannot be analyzed.

Accordingly, there is a need to develop a new bioassay apparatus that could solve the problem of photobleaching, reduce background and light scattering that is characteristic of conventional light sources, e.g., UV light, and enhance emission from target analytes. The use of upconverting nanoparticles as labels to overcome the problem of weak emission, fluorophore bleaching, and background autofluorescence from the sample is described in greater detail below. One objective of the present invention is to enhance the emission from upconverting nanoparticles in conjunction with resonant grating structures to achieve a high sensitivity biosensing platform for detecting analytes, such as, but not limited to proteins, pathogens, and electrolytes in body fluids. The enhanced emission of upconverting nanoparticles is important since the lower background signal and higher sensitivity of upconverting nanoparticles over traditional fluorescent dyes, quantum dots, or other fluorescent labels lead to superior performance in measurement accuracy in a bioassay, for example but not limited to immunoassay applications.

In one aspect, the present invention provides a bioassay system for detecting target analytes in body fluids, e.g., blood, serum, plasma, synovial fluid, cerebrospinal fluid, and urine. In general, the bioassay system comprises two parts: (i) a biosensor such as a waveguide comprising a resonance grating structure, the resonance grating structure defining an enhancement region which extends from the surface and along a direction normal to the surface for a predetermined distance, and a secondary antibody directed to the target analyte bound to the surface of the resonance grating structure through a linkage chemistry; and (ii) an upconverting nanoparticle conjugated to a first antibody directed to an epitope of the target analyte that differs from the epitope to which the secondary antibody is directed. The upconverting nanoparticle comprises an optical property that absorbs infrared light and emits visible light in response to absorption of the infrared light.

In one embodiment of the system described herein, the target analyte is bound to an anti-target analyte antibody conjugated to an upconverting nanoparticle, for example, NaYF₄:(Yb,Er,Tm), NaYbF₄:(Yb,Er,Tm), CaF₂:(Yb,Er), La₂O₃:(Yb,Er), The target analyte is also bound to a second anti-target analyte antibody (that differs from the first anti-target analyte antibody) that is coupled to the surface of the resonance grating structure through a linking chemistry, for example, through strepavidin. Accordingly, the target analyte is “labeled” by a nanoparticle by the first antibody, and “captured” to the surface of the resonance grating structure by the second antibody. At the surface of the resonance grating structure, the target analyte is optically detected.

In one embodiment, the bioassay sensor further comprises a highly refractive layer (e.g., TiO₂ and Ta₂O₅) disposed on the surface of the resonance grating structure, the highly refractive layer having a refractive index of at least 1.5. The resonance grating structure comprises a photonic crystal tuned to a predetermined resonance condition and an antibody coupled to the resonsance grating structure through a linkage chemistry. In an alternative embodiment, the present invention provides a bioassay sensor, comprising a waveguide comprising a resonance grating structure; and an antibody coupled to a surface of the resonance grating structure through a linkage chemistry.

In another aspect, the present invention is directed to a composition of matter comprising an antibody and an upconverting nanoparticle bound to the antibody.

In one embodiment, the composition of matter further comprises a target analyte bound to a binding site of the antibody.

In yet another aspect, the present invention is directed to an immunoassay kit comprising a device having a resonance grating structure and a second antibody coupled to a surface of the resonance grating structure through a linkage chemistry; and a composition of matter having an upconverting nanoparticle conjugated to a first antibody, wherein the first antibody and the second antibody are directed to the same target analyte.

In yet another aspect, the present invention is directed to a method for detecting a target analyte in a body fluid. A resonance grating structure having a plurality of capturing sites is provided. An optical illumination is provided to the resonance grating structure which has one or more target analytes coupled to an upconverting particle captured via an antibody at the capturing site. Optical responses from the capturing sites are detected and are indicative of the presence of the target analyte at the corresponding capturing sites.

As used herein, the term coupled means at least two elements joined together directly or indirectly.

### BRIEF DESCRIPTION OF THE DRAWINGS

FIG. 1A schematically illustrates a sectional view and FIG. 1B illustrates a perspective view of a resonant waveguide having a grating structure in accordance with one embodiment of the present invention.

FIG. 2 schematically illustrates the electric field on the surface of a substrate under a resonance condition in accordance with one embodiment of the present invention.

FIG. 3 schematically illustrates a method for sensing analytes using a bioassay system, such as an immunoassay, in accordance with one embodiment of the present invention.

FIG. 4 schematically illustrates a method for sensing analytes using a bioassay system, such as an immunoassay, in accordance with another embodiment of the present invention.

### DESCRIPTION

One approach of increasing bioassay sensitivity is to increase the light level of the illumination by using a polymer-based grating substrate to boost the emission, but this can lead to certain problems, such as high background and increased sample temperature and sample damage. For example, in such a bioassay system, the polymer-based grating substrate begins to present excessive amount of background due to autofluorescence. This interference from the background is not suitable for high sensitivity biosensing applications, for example, immunoassays. High quality and purity quartz substrates have been used to address this problem, but these materials have led to much higher material cost and more complicated manufacturing processes. Accordingly, the present invention employs upconverting nanoparticles that absorb infrared excitation in conjunction with resonant grating structures to achieve high sensitivity in a biosensing platform for detecting analytes.

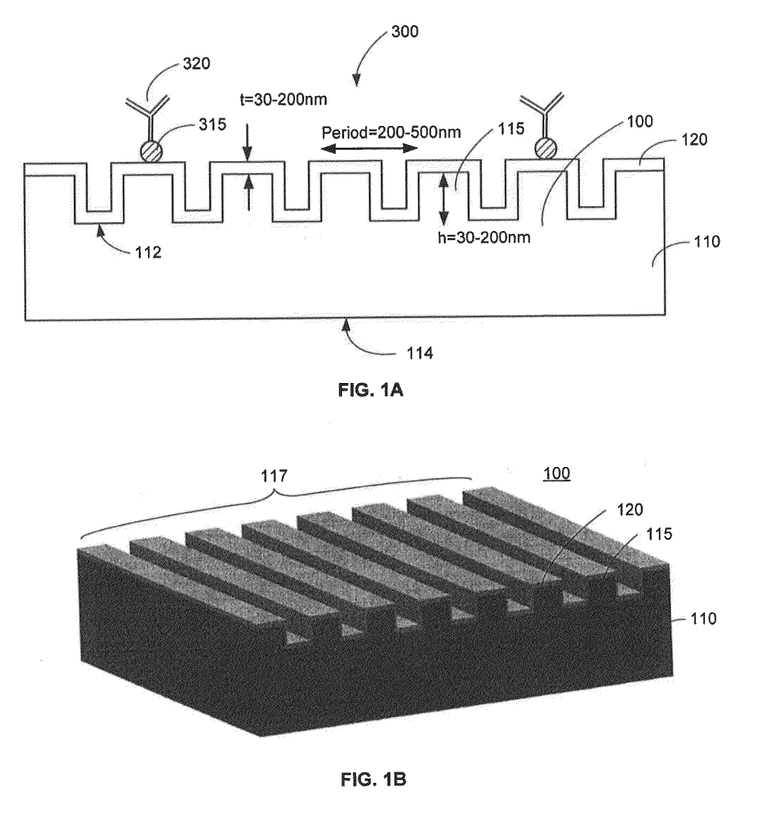

Referring to FIGS. 1A and 1B, FIG. 1A illustrates a sectional view and FIG. 1B illustrates a perspective view of a sensor 300 comprising a resonant waveguide 100 in accordance with one embodiment of the present invention and a secondary antibody 320 directed to a target analyte, the secondary antibody being bound to the surface of the resonant waveguide 100 through a chemical linkage 315. In one embodiment, the chemical linkage 315 comprise a binding protein, such as streptavidin.

With continued reference to FIGS. 1A and 1B, resonant waveguide 100 comprises a substrate 110 having a grating structure 115 formed on a surface 112 of the substrate 110, and a refractive layer 120 formed on the surface 112 of grating structure 115. In one embodiment, one or more additional refractive layers (not shown) may be formed on refractive layer 120.

With continued reference to FIG. 1A, in one embodiment, substrate 110 is made of an optically transparent material or a polymeric material (for example, but not limited to, polystyrene, ultraviolet curable polymer or glass), and grating structure 115 is formed to have a grating period of about 360 nm, a grating groove of about 50 nm in depth, and a duty cycle of about 36%. It is appreciated that the grating period ranges from about 200 nm to about 500 nm; the grating groove depth ranges from about 30 nm to about 300 nm; the thickness of refractive layer 120 (having a refractive index of greater than 1.5) ranges from about 30 nm to about 200 nm; and the duty cycle ranges from about 30% to about 50%. The substrate 110 is transparent and compatible with the near-infrared (NIR) excitation and visible light detection of emission from upconverting nanoparticles. The grating material is made from, for example, but not limited to, SiO₂, polymeric material such as polystyrene, silicone, thermoplastics, or glass such as fused silica and quartz.

In one embodiment, refractive layer 120 can be made of a high refractive material (e.g., TiO₂ with a refractive index of about 2.35, or Ta₂O₅ with a refractive index of about 2.09). It is appreciated that various forms and configurations of the grating are possible to produce a resonance mode, and can enhance the excitation of upconverting nanoparticles. For example, in one embodiment of the invention, the grating has more than one layer of thin coating of high refractive material, and enhances the intensity of excitation light when nanoparticles are close to the surface 112 (in the range of about 1-150 nm, preferably in the range of about 1-2000 nm, more preferably below 300 nm). The resulting surface of grating structures 115 and refractive layer 120 behave much like a grating, but tuned to specific resonance modes and wavelengths where the enhancement occurs. At the resonance wavelength, the evanescent electrical field on or above the substrate surface 112 is very high (e.g., greater than 50-fold than without the grating) so surface labels such as upconverting nanoparticles can emit strongly. Although refractive layer 120 is shown and described in FIG. 1A, it is to be understood that resonant waveguide 100 may still constitute a photonic crystal, without the presence of refractive layer 120.

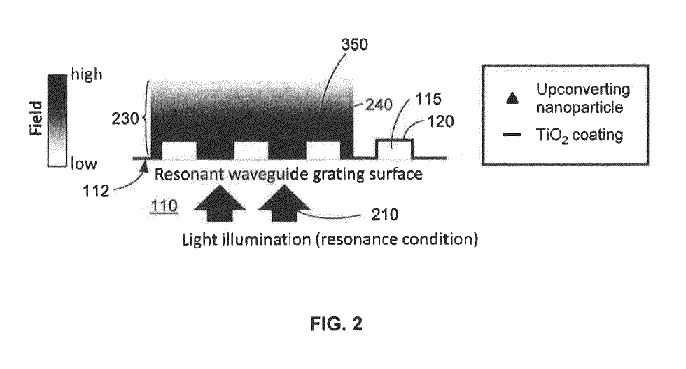

FIG. 2 schematically illustrates the electric field on the surface of a substrate 110 under a resonance condition in accordance with an embodiment of the invention. As shown in FIG. 2, a light beam 210 impinges from a surface 114 (see FIG. 1A) of substrate 110 opposite to the surface 112. A plurality of upconverting nanoparticles 350 are bound to the surface 112 of grating structure 115, on which a refractive layer 120 is formed. In one embodiment, the upconverting nanoparticles 350 are conjugated to an antibody (not shown) that is directed to a target analyte (not shown). The target analyte is bound to the nanoparticle conjugated antibody and captured at the surface 112 by a second antibody (not shown) directed to the target analyte. The second antibody is bound to the surface 112 through a linkage chemistry 240 (for example, but not limited to, streptavidin, surface couplings via reactive functional groups such as amino, hydroxyl, thiol, and carboxyl groups, modified DNA probes, and peptides).

Referring to FIGS. 1A, 1B, and 2, in one embodiment, the wavelength of light beam 210 directed to surface 114 of substrate 110 matches a resonance condition of the grating structure 115 that constitutes a photonic crystal. As a result, the intensity of light beam 210 is greatly enhanced at an enhancement region 230 over a textured surface 117 of substrate 110. In this embodiment, textured surface 117 is formed of grating structure 115 (parallel ridges and valleys), as shown in FIG. 1B. It is appreciated that, in other embodiments, textured surface 117 may be formed of an array of protrusions (e.g., pillars and rods), or an array of recesses (e.g., circular recesses, rectangular recesses, and hexagonal recesses). In an ideal situation, the enhancement of the light beam intensity can be as high as 1500-fold with respect to the intensity of light beam 210 without using a photonic crystal. In one embodiment, the enhancement is less and may reach around 50-fold.

Other materials useful for enhancing the emission are, for example, plasmonic surface structures made from metallic films. In one example, the emission of upconverting nanoparticles is enhanced almost 300-fold, using plasmonic nanoantenna (gold dots on pillar structures). The advantage of this embodiment is that the upconverting nanoparticles/labels are more robust and do not photobleach and last a long time compared to conventional fluorophores (e.g., several hours versus a few minutes for conventional fluorophores). However, the plasmonic structures are in general more difficult to manufacture and may have several drawbacks, such as strong light absorption that can cause heating effects. A transparent dielectric material, used in yet another embodiment of the invention, avoids this problem.

In one embodiment, a high sensitivity system for biomedical assay sensing applications includes two components. For example, a first component is a sensor comprising a resonance grating structure made from a transparent dielectric material, such as a photonic crystal waveguide, and a secondary antibody directed to a target analyte that is bound to the surface of the resonance grating structure. In a preferred embodiment, a second component comprises an upconverting nanoparticle bound to a primary antibody directed to the same target analyte as the secondary antibody. Conjugates other than an upconverting nanoparticle are also contemplated by the invention, for example, a conventional fluorophore or a downconverting nanoparticle. The system is designed for high sensitivity assay applications to provide low cost and ease of mass production and minimal background signal.

With continued reference to FIG. 2, the system of the present invention has improved sensitivity with the enhanced fields at enhancement region 230. The upconverting particles 350 are located in the enhancement region 230 of the resonance grating structure 100 which extends for more than a hundred nanometers from surface 112 of substrate 110. It is appreciated that enhancement region 230 has a brightline boundary at surface 112, but a rather blurry boundary as the electric field Which gradually diminishes away from surface 112. In one embodiment, the blurry boundary may be defined as a contour line where the electric field is half of the strongest electric field proximate surface 112. The emission may be enhanced by about 50-fold or more, to improve the assay sensitivity.

The sensor of the present invention has numerous advantages over conventional immunoassay systems. First, the present invention does not suffer from intensive background signal emanating from autofluorescence. Grating structure 115 has a grating period, a grating groove depth, a duty cycle, such that, in one embodiment of the invention the resonance of grating 115 is tuned to the 980 nm absorption peak of the upconverting nanoparticles 350 and to normal incidence of the illuminating light source 210, In this embodiment, there is substantially no background from the substrate 110 because the 980 nm light does not cause autofluorescence from grating 115 or from the body fluid sample being analyzed.

The bioassay system according to the invention has additional advantages over conventional bioassay systems, such as immunoassay systems. For example, the sensor of the present invention does not suffer from photobleach of fluorophores. Upconverting nanoparticles 350 are known to be very stable and do not photobleach and can better handle the high electric fields proximate surface. Conventional fluorophores are less desirable and photobleach quickly.

Additionally, the sensor of the present invention has enhanced localized surface emission. Upconverting nanoparticles 350 have nonlinear absorption dependence with respect to the illumination intensity. Because of this nonlinear property, only bound nanoparticles, or those that are close to the surface, are excited selectively. As a result, there is little emission from the surrounding media.

Further, the sensor of the present invention is applicable in homogeneous assay applications. Because of the enhanced surface-emission of bound nanoparticles, the assay washing step can be eliminated leading to a much simpler system design.

Moreover, the sensor of the present invention does not require focused light illumination, In the invention disclosed herein, a laser (or a coherent light source) is not needed since the enhancement effect from the resonance grating increases the intensity without the need for focusing and high power of lasers. Low-cost incoherent sources, for example, but not limited to, LED, gas discharge lamps, and high-intensity discharge lamps can be used instead of a laser,

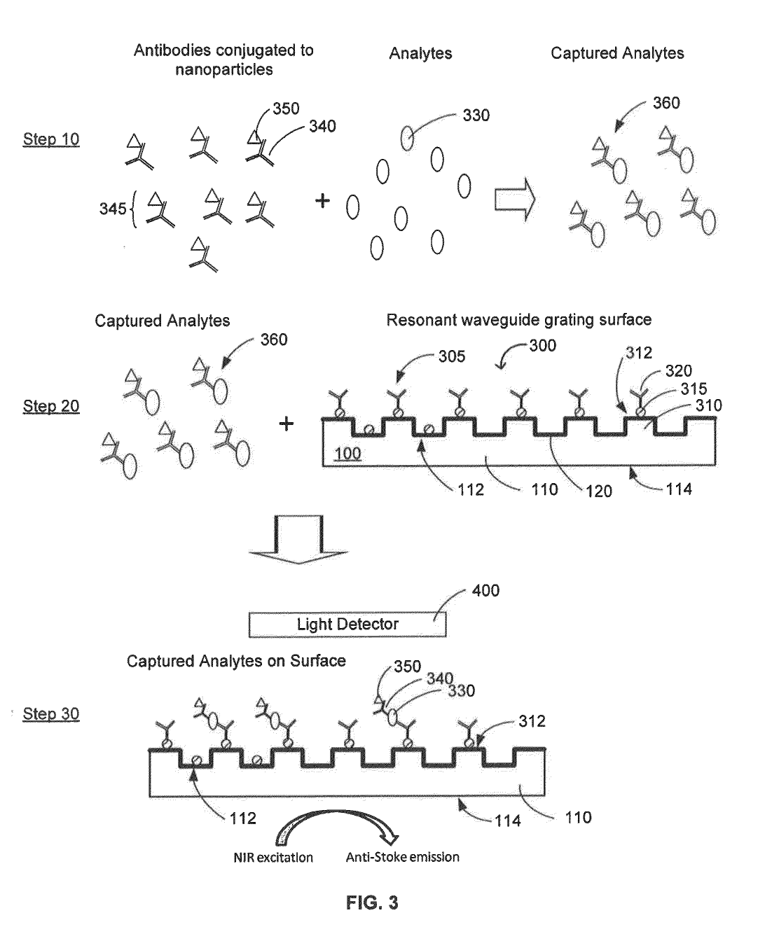

FIG. 3 schematically illustrates a method for detecting analytes using a bioassay apparatus in accordance with one embodiment of the present invention. In Step 10, as composition of matter 345 comprising a nanoparticle 350 conjugated to a first antibody 340 targeted to as target analyte 330 is mixed with a body fluid such as blood, serum, plasma, synovial fluid, cerebrospinal fluid, or urine in which the target analyte 330 is suspected to be present. In one embodiment, the composition of matter 345 is formed by mixing nanoparticles 350 and first antibodies 340 directed to the target analyte in an organic or inorganic solvent for coupling nanoparticles to antibodies. In other embodiments, the nanoparticle labeled antibodies are a powder form that is mixed in the fluid containing the suspected analytes 330 to be detected.

In the next step, Step 20, nanoparticle labeled antibodies 345 bound to analytes 330 are applied to a bioassay sensor 300 having a resonant waveguide grating surface 112. In one embodiment, bioassay sensor 300 comprises a substrate 110 having a grating structure 310, a refractive layer 120 formed on the surface of grating structure 310, and second antibodies 320 directed to the target analyte 330 coupled and immobilized to a surface 312 of grating structure 310. In one embodiment, each grating period of grating structure 310 comprises an anti-target antibody coupled therewith to constitute a target analyte capturing site 305. In one embodiment, second antibodies 320 are immobilized on grating structure 310 through linkage chemistry 315 (e.g., streptavidin) to capture target analytes 330 coupled to, for example, a nanoparticle selected from the group consisting of NaYF4: Yb—Er, CaF2:Yb,Er, NaYbF4:Ho,Tm,Er from the body fluid of a patient being analyzed. Captured analytes 360 are bound to second antibodies 320 that are directed to the same target analytes 330 and are coupled to the surface 312 of grating structure 300. In one embodiment, bioassay sensor 300 is washed by pure water to remove uncaptured analytes from substrate 110. Bioassay sensor 300 is now ready for optical examination.

In Step 30, light, e.g., a near-infrared (NIR) light beam having a wavelength of about 980 nm is applied to a surface 114 of substrate 110 that is on the side of the substrate 110 of grating structure 300 opposite to the surface 312 which may include refractive layer 120. Because the NIR light beam is chosen to match the resonance condition of grating structure 310, the upconverting nanoparticles 350 conjugated to the first antibody 340 directed to the target analyte 330 and bound to the grating surface 312 through the second antibody 320 also directed to the target analyte 330 are excited by an enhanced NIR excitation. Depending on the optical property of upconverting nanoparticles 350, a light beam (such as visible light) is emitted in response to the enhanced NIR excitation. The presence and/or absence of analytes 330 on capturing sites 350 is determined using a light detector 400.

In one embodiment, an upconverting nanoparticle 350 is conjugated to a antibody 340 in a “sandwich” assay to detect the captured target analyte 330 bound to a second antibody directed to the target analyte 330 at capturing sites 305 on the surface of a resonant grating structure 310. The resonance of grating structure 310 is tuned to the peak of their absorption peak to have the largest enhancement effect and to improve the assay sensitivity.

In another embodiment, the bioassay sensor is used in a “blocking assay” application where binding of particles to the surface of the resonant grating structure 310 leads to “detuning” of the resonance mode and reduction of the enhancement effect. The surface binding events result in change of the refractive index of refractive layer 120 and are enough to detune the resonance away from the illumination wavelength. For example, a laser with a very narrow wavelength bandwidth and a grating with a high quality factor resonance structure, or a very narrow and sharp resonance peak, is a very sensitive arrangement for detecting changes to the refractive index at the surface. As the resonance gets detuned away from the laser wavelength, a sharp drop in the enhanced emission of the upconverting nanoparticles may be observed.

In yet another embodiment, the sensing system of the present invention takes advantage of the relatively narrow enhancement region where the enhancement takes place and is useful to detect binding kinetics and follow changes in the quantity of target analyte over time. Not to be bound by theory but it is believed that the emission from upconverting particles captured at the surface of the grating structure is confined to less than a 100 nm region proximate the grating surface. Any upconverting nanoparticle labels outside the 100 nm region are not excited and consequently have much less emission than nanoparticles that are positioned closer to the surface of the grating structure and within the 100 nm region.

For example, in one embodiment, the grating surface 312 is first pretreated with labels (such as fluorophores or nanoparticles) by predefined linkage elements (such as thrombin) that are susceptible to cleavage by the action of a target protein. When exposed to thrombin, the labels are cleaved at the linkage element and diffuse away from the enhancement region. Thus, cleavage of the linkage element frees the label away from the surface in the 100 nm region above the grating structure and the label cannot be detected. Decreased emission allows for quantitative determination of a target protein, such as thrombin. In another example, the labels can specifically bind to the surface 312 and increase the signal indicating the binding events.

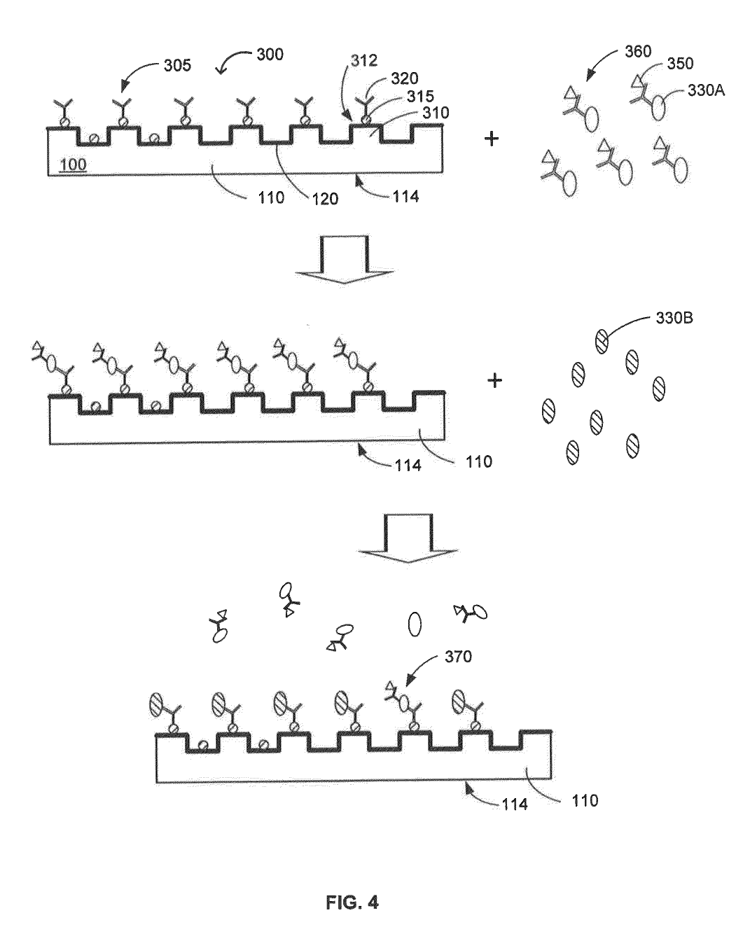

In another embodiment of the invention, as shown in FIG. 4, the bioassay system is configured in a competitive heterogeneous immunoassay mode where a target analyte 330A, for example, is bound to an upconverting nanoparticle to form a nanoparticle labeled conjugated analyte 360. The target analyte 330B in the patient's body fluid sample is unlabeled. The nanoparticle labeled conjugated analyte 360 is then applied to bioassay sensor 300, such that capturing sites 305 are bound with a conjugated analyte 360. Then, the patient's body fluid including unlabeled analytes 330B undergoing analysis is applied to the resultant “labeled” bioassay sensor 300. The patient's unlabeled target analyte 330B competes for binding sites 305 on the grating surface-coupled anti-target analyte antibody 320 with the nanoparticle labeled target analyte 360. Upon introduction of the patient's unlabeled body fluid target analyte 330B, the labeled target analytes 360 are released from the grating surface 312 and are free to diffuse into the solution away from the enhancement region 230 (see FIG. 2). Detectable light emission arising from the nanoparticle labeled target analyte 360 decreases in the presence of patient unlabeled analyte 330B. This configuration is commonly used in drug analysis and in clinical biochemistry of hormones and proteins. In this mode, unlabeled analyte 330B in the patient sample competes with nanoparticle-labeled analyte 360 at the enhancing surface 312. The unbound analyte is washed away, and the remaining labeled analyte 370 bound to the grating surface is measured. A decrease in emission of the labeled bound analyte 360 is proportional to the amount of target analyte in the patient's body fluid sample.

Although embodiments of the present invention have been described in detail, it is to be understood that these embodiments are provided for exemplary and illustrative purposes only. Various modifications and changes may be made by persons skilled in the art without departing from the spirit and scope of the present disclosure as defined in the appended claims.

What is claimed is:

1. A bioassay system for detecting a target analyte, comprising:
a waveguide comprising a resonance grating structure having a grating surface, the resonance grating structure defining an enhancement region;
a first antibody coupled to the enhancement region directed to the target analyte;
an upconverting nanoparticle; and,
a second antibody coupled to the upconverting nanoparticle directed to the target analyte.

2. The system of claim 1, wherein the enhancement region extends from the grating surface and along a direction normal to the grating surface for a predetermined distance.

3. The system of claim 1, wherein the upconverting nanoparticle comprises an optical property that absorbs infrared light and emits visible light in response to absorption of the infrared light.

4. The system of claim 1, wherein the upconverting nanoparticle is coupled to the grating surface through the target analyte to be detected.

5. The system of claim 4, wherein the upconverting nanoparticle is coupled to the target analyte through the second antibody, and the target analyte is coupled to the grating surface through the first antibody.

6. The system of claim 5, wherein the first antibody is coupled to the surface of the resonance grating structure through a chemical linkage.

7. The system of claim 1, further comprising a refractive layer on the grating surface having a refractive index of at least 1.5.

8. The system of claim 1, wherein the resonance grating structure comprises a photonic crystal tuned to a predetermined resonance condition.

9. A method for detecting a target analyte in a body fluid, comprising;
providing a resonance grating structure comprising a plurality of capturing sites;
applying an optical illumination to the resonance grating structure, the resonance grating structure having one or more target analytes captured at the capturing sites through a first antibody directed to the target analyte, each target analyte being coupled to an upconverting nanoparticle; and
detecting optical responses from the capturing sites, an optical response from one of the capturing sites being indicative of the presence of the target analyte at that capturing site.

10. The method of claim 9, further comprising:
prior to applying the optical illumination
washing the resonance grating structure to remove uncaptured target analytes.

11. The method of claim 9, further comprising:
mixing a first solution containing said upconverting nanoparticles coupled to a secondantibody directed to the target analyte with a second solution comprising the body fluid suspected of containing the target analyte to form a third solution;
waiting for a predetermined time period to allow coupling of the nanoparticle labeled antibody to the target analyte in the third solution; and
applying the third solution to the capturing sites.

12. An apparatus for detecting a target analyte, comprising:
a resonance waveguide grating structure comprising a plurality of capturing sites, the resonance waveguide grating structure defining an enhancement region that extends from a surface of the capturing sites and along a direction normal to the surface for a predetermined distance;
a refractive layer disposed on the capturing sites;
a second antibody directed to said target analyte positioned at the enhancement region and coupled to the capturing sites; and
a first antibody directed to said target analyte positioned at the enhancement region coupled to a plurality of upconverting nanoparticles, wherein said first antibody is coupled to said second antibody through said target analyte;
a light source for generating an optical signal of a first wavelength to excite the upconverting nanoparticles; and
a light detector for sensing an optical response of a second wavelength, wherein the second wavelength is shorter than the first wavelength.

13. The apparatus of claim 12, wherein the first wavelength matches a resonance condition of the waveguide grating structure.

14. The apparatus of claim 12, wherein the first antibody is respectively coupled to the capturing sites through a linkage chemistry.

15. A bioassay system for detecting a target analyte, comprising:
a waveguide comprising a resonance grating structure; and
an antibody to the target analyte coupled to a surface of the resonance grating structure through a linkage chemistry.

16. The bioassay system of claim 15, further comprising a refractive layer on the surface of the resonance grating structure, the refractive layer having a refractive index of at least 1.5.

17. The bioassay sensor of claim 15 further comprising an antibody directed to a target analyte conjugated to an upconverting nanoparticle.

18. The bioassay system of claim 15, wherein the resonance grating structure comprises a photonic crystal selected from the group consisting of replicated gratings, holographic photonic crystal, and porous silicon photonic crystal, tuned to a predetermined resonance condition.

19. A composition of matter comprising an antibody directed to a target analyte conjugated to an upconverting nanoparticle.

20. The composition of matter of claim 18 further comprising the target analyte bound to a binding site of the antibody.

21. An immunoassay kit for detecting a target analyte, comprising:
a bioassay system having a resonance grating substrate and a first antibody coupled to a surface of the resonance grating substrate through a linkage chemistry; and
a composition of matter having a second antibody conjugated with an upconverting nanoparticle, wherein the first and second antibodies are directed to different epitopes of the same target analyte.
